(19) United States
(12) Patent Application Publication
(10) Pub. No.: US 2025/0305008 A1
(43) Pub. Date: Oct. 2, 2025

(54) COMPOSITIONS AND METHOD FOR MAKING SUCCINIC ACID

(71) Applicant: PORUS BIOSCIENCES LLP [IN]/[IN],, Hyderabad (IN)

(72) Inventors: Prashant GAUR, Hyderabad (IN); Raviprasad PODILI, Hyderabad (IN); Venkata Naga JALADI, Hyderabad (IN)

(73) Assignee: PORUS BIOSCIENCES LLP [IN]/[IN],, Hyderabad (IN)

(21) Appl. No.: 19/210,259

(22) Filed: May 16, 2025

(30) Foreign Application Priority Data
Nov. 24, 2023 (WO) PCT/IN2023/051092

(51) Int. Cl. C12P 7/46; C12M 1/00; C12M 1/34; C12N 1/20; C12R 1/15

(52) U.S. Cl. CPC C12P 7/46; C12M 29/18; C12M 29/20; C12M 41/34; C12M 47/02; C12N 1/205; C12R 2001/15

(57) ABSTRACT

The present invention discloses an engineered Corynebacterium strain and a process of making succinic acid in microaerobic conditions. The process of making succinic acid as disclosed in the present invention utilizes carbon dioxide as secondary carbon source during production phase and replaces bicarbonate as a source of secondary carbon. Further, integration of gas recycle and the biomass recycle in the process made it sustainable while having minimal or no loss in specific productivity for succinic acid production. The present invention has the advantage of yielding higher succinic acid levels while reducing resource costs.

### CROSS-REFERENCE TO RELATED APPLICATIONS

This U.S. nonprovisional application claims priority to International Application No. PCT/IN2023/051092, filed on Nov. 24, 2023.

### FEDERALLY SPONSORED RESEARCH AND DEVELOPMENT

NA

### FIELD OF THE INVENTION

The present invention is related to a Corynebacterium strain and a process of making succinic acid in microaerobic conditions by using carbon dioxide as a secondary carbon source during the production phase. The invention more specifically relates to a system for making succinic acid by using carbon dioxide as a secondary carbon source during the production phase, and recycling carbon dioxide and biomass for sustainability.

### BACKGROUND OF THE INVENTION

Succinic acid is an alpha-omega dicarboxylic acid and is an industrially important platform chemical. It has role in multiple industries, including medicine, food and nutrition, as a building block for other molecules.

Succinic acid is an intermediate of TCA cycle and is one of the end products of anaerobic metabolism during fermentation of many microorganisms.

In bio-based succinic acid production process employing microorganisms that operate the reductive branch of TCA cycle, carbon dioxide (CO2) is fixed to form succinic acid. Thus, by being based on renewable resources, bio-based succinic acid production is a much more eco-friendly process when compared to the petrochemical process.

Succinic acid production through bio route using non-GM and GM microbes has been commercialized with little economic success owing to high manufacturing cost primarily driven by lower yields, productivity and higher input costs. Corynebacterium strains are gram positive microbial strains that grow rapidly and have been used commercially for producing various organic acids.

Wildtype Corynebacterium strains give a poor yield of succinic acid (SA). Thus, to enhance the succinic acid yield, (g-SA/g-Glucose), conventionally known methods have undertaken the approach of deleting Lactate Dehydrogenase, ldh gene, while overexpressing Pyruvate Carboxylase, (pyc gene).

Traditionally bicarbonate salts are often used as a secondary carbon source for organic acid production including succinic acid production and are the second most expensive raw material amongst the fermentation components. Cao et al (Ref 1) has shown a bioprocess for converting CO2 into succinic acid (SA) Actinobacillus succinogenes by an integrated fermentation and membrane separation process. There are multiple challenges linked with membrane usage, such as high costs and membrane replacement requirement. The current invention provides a low cost, high yield giving system, and method for producing SA using CO2, and recycling CO2 and biomass both. This process is highly amenable to scaling up.

The present invention addresses these gaps of lower yields, lower productivity and higher cost of production associated with succinic acid production, by means replacing or supplementing bicarbonate as source of secondary carbon and recycling microbial biomass and carbon dioxide for production of succinic acid.

### SUMMARY OF THE INVENTION

The current invention encompasses a system and a method for producing succinic acid (SA) sustainably with high yield. The invention encompasses a system and method for producing SA using CO2 as the secondary carbon source, wherein the CO2 and the biomass is recycled without having a significant negative impact on the yield of SA.

One embodiment of the current invention is a self-sustainable system for producing succinic acid, the system comprising of:
fermentation vessel (101) comprising microbial biomass,
a gas inlet (103) for sparging gas into the vessel;
a gas outlet (105) for the exhaust gas from the vessel, wherein the exhaust gas is sparged back into the vessel after passing through a gas compressor (107) to recycle the CO2;
wherein the gas sparged through the inlet is either 100% air or air mixed with 5-35% v/v of CO2 and wherein the CO2 is the secondary carbon source for succinic acid production.

In one embodiment, the pressure of gas sparged through the gas inlet (103) is 0.05 to 2 bars.

In one embodiment, CO2 is used instead of carbonate or bicarbonate salts as the secondary carbon source for succinic acid production.

In one embodiment, the biomass in the fermenter vessel is present at 5-8 g dry cell weight/litre (dcw/L) in the fermentation vessel.

In one embodiment, liquified corn mash is used as the primary carbon source for the succinic acid production.

In one embodiment, the inlet gas is sparged at the rate of 0.03-0.11 vvm (litres/liters/min), wherein vvm is Volume of gas sparged per unit Volume of broth per Minute, Calculated by dividing the gas flow (L/m) by the Volume (L) of broth.

In one embodiment, a gas analyzer is attached to monitor the inlet and exhaust gas composition.

In one embodiment, a cell separation module (109) is connected to the fermentation vessel to separate the broth containing the succinic acid from the biomass for recycling the separated biomass into the fermentation vessel for succinic acid production.

In one embodiment, the cell separation system comprises a filtration unit or a centrifugation unit.

In one embodiment, the system comprises more than one fermentation vessels and wherein the exhaust gas from one vessel is sparged through the gas inlet of a second vessel at a pressure of 0.05 to 2 bars.

In one embodiment, the yield of succinic acid using the system disclosed herein is 0.7-0.9 g/g glucose.

One embodiment of the current invention is a method for producing succinic acid in a self-sustainable system, the method comprising the steps of:
Culturing LDH-deficient Coryneform bacterium in a fermentation vessel up to biomass of 5-8 g-dcw/L in 100% air;
Initiating succinic acid production by sparging 0.03-0.11 vvm of air mixed with 5-35% v/v CO2 gas into the vessel with the biomass grown in step (a), via a gas inlet;
recycling the exhaust gas from the gas outlet of the fermentation vessel back into the vessel via the gas inlet after passing through a gas compressor to obtain inlet pressure of 0.05 to 2 bars, to maintain SA production.

In one embodiment, the pressure of gas sparged through the gas inlet (103) is 0.05 to 2 bars. In one embodiment, CO2 is used instead of carbonate or bicarbonate salts such as sodium or potassium or ammonium or magnesium or calcium salts as the secondary carbon source for succinic acid production in the method disclosed herein.

In one embodiment, liquified corn mash is used as the primary carbon source for the succinic acid production in the method disclosed herein.

In one embodiment, a gas analyzer is attached to monitor the inlet and exhaust gas composition in the method disclosed herein.

In one embodiment, cell separation module (109) is connected to the fermentation vessel to separate the broth containing the succinic acid from the biomass for recycling the separated biomass into the fermentation vessel for succinic acid production in the method disclosed herein.

In one embodiment, the cell separation system comprises a filtration unit or a centrifugation unit in the method disclosed herein.

In one embodiment, the system comprises more than one fermentation vessels and wherein the exhaust gas from one vessel is sparged through the gas inlet of a second vessel at a pressure of 0.05 to 2 bars in the method disclosed herein.

In one embodiment, the yield of succinic acid is 0.7-0.9 g/g glucose by the method disclosed herein.

### BRIEF DESCRIPTION OF DRAWINGS

FIG. 1 shows a schematic of the system configuration showing the use of CO2 as the secondary carbon source, and recycling of the CO2. The schematic shows a fermentation vessel (101), gas inlet (103), gas outlet (105), and gas compressor (107).

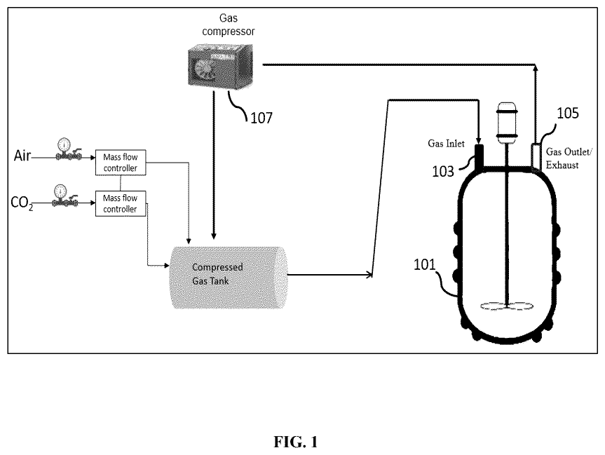

FIG. 2 shows a schematic of the system configuration use of CO2 as the secondary carbon source, and recycling of the CO2 and recycling of the biomass. The schematic shows a fermentation vessel (101), gas inlet (103), gas outlet (105), and gas compressor (107) and a cell separation unit (109).

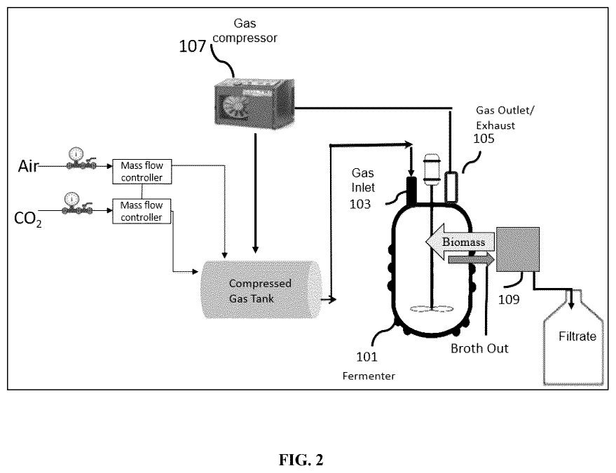

FIG. 3 shows a schematic of more than one fermentation vessels, the connected fermentation vessels using recycled biomass and CO2. The figures shows two vessels being connected for recycling of CO2 and biomass. The second vessel uses recycled CO2 from the first fermentation vessel, wherein the exhaust from the first vessel is connected to the gas inlet of the next vessel. The figure also shows biomass recycling back into the same vessel after passing the fermentation broth through a cell separation unit (109). The clarified broth containing.

### DETAILED DESCRIPTION OF THE INVENTION

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that other changes may be made without departing from the scope of the embodiments. The following detailed description is therefore not to be taken in a limiting sense.

The current invention encompasses a system and a method for producing succinic acid (SA) sustainably with high yield. The invention encompasses a system and method for producing SA using CO2 as the secondary carbon source, wherein the CO2 and the biomass is recycled without having a significant negative impact on the yield of SA.

Succinic acid is an alpha-omega dicarboxylic acid and is one of the most important platform chemicals, and has role in multiple industries, including medicine, food and nutrition, as a building block for other molecules such as 1,4-butanediol (BDO), tetrahydrofuran, and gamma-butyrolactone. It is considered as one of the “Top Value-Added Chemicals from Biomass” by US Department of Energy (DOE). Platform chemicals are substances that serve as starting materials for the manufacture of multiple value-added products for a wide range of applications. Hence platform chemicals are able to create great commercial value. Many studies are being done to find more bio-based, sustainable ways to produce these platform molecules.

Succinic acid is conventionally made using petrochemicals, which is not sustainable. Bio-based methods of producing succinic acid are sustainable, but not cost-effective due to high cost of raw materials and biomass production, such as bicarbonate salts, which are usually used as secondary carbon sources for succinic acid production by fermentation.

Succinic acid production through bio route using non-genetically modified and genetically modified microbes has been commercialized with little economic success owing to high manufacturing cost primarily driven by lower yields, productivity and higher input costs. Corynebacterium strains are gram positive microbial strains that grow rapidly and have been used commercially for producing various organic acids.

Production of succinic acid for industrial processes is mainly through petrochemical processes by hydrogenation of butane. Succinic acid is also chemically produced by catalytic hydration of maleic acid anhydride to succinic acid anhydride and subsequent water addition or by direct catalytic hydration of maleic acid. These processes are neither environment-friendly nor cost-effective. Production of succinic acid by fermentation is an alternative process (bio-based process), in which a carbon source and a sugar source are used. It is generated via the tricarboxylic acid cycle (TCA) in cells. Sugar is usually the primary carbon source.

Since SA is an acidic product, a large amount of alkaline neutralizers are required to maintain the pH during SA biosynthesis. Traditionally, during SA production, carbonate or bicarbonate salts are used for maintaining pH and also as secondary carbon source. But these carbonate or bicarbonate salts, which can be potassium or ammonium or magnesium or calcium salts such as NaHCO3, MgCO3 which can neutralize the pH and also provide carbon for feeding into the TCA cycle for SA production, are very expensive. Moreover, many of these salts are not very soluble in water which makes scaling up of SA production by bio-based or fermentation methods highly cost-ineffective and unsustainable.

Traditionally bicarbonate salts are often used as a secondary carbon source for organic acid production including succinic acid production and are the second most expensive raw material amongst the fermentation components.

Moreover, when carbonate or bicarbonate salts such as NaHCO3 or MgCO3 are used as the neutralizing agents with continuously sparging pure CO2 (Ref 2: Herselman et al., 2017), less exogenous CO2 is used in the SA biosynthesis since intrinsic bicarbonate or carbonate ions in the salts are preferentially consumed. Moreover, the unfixed CO2 in this process can become a pollutant when released into the atmosphere, especially in the industrial production process.

The current study uses CO2 as the secondary carbon source in a renewable manner, either without using the carbonate or bicarbonate salts totally; or by supplementing the bicarbonate salts with recycled CO2 as the secondary carbon source, without decreasing the yield.

The current invention also encompasses recycling of bacterial biomass up to four times, which makes the system more sustainable and minimizes the problem of product inhibition, without causing major decreases in yield.

Some studies have shown biomass recycling using a membrane bioreactor, but the ingredients of the fermentation medium could not be fully consumed in a membrane bioreactor.

The present invention addresses these gaps of lower yields, lower productivity and higher cost of production associated with succinic acid production., by means of strain engineering and replacing bicarbonate by carbon dioxide as source of secondary carbon and recycling microbial biomass for production of succinic acid using liquified corn mash.

### Definitions

The term “fermentation” as used herein has its ordinary meaning as known to those skilled in the art. “Fermentation” includes culturing of a microorganism or group of microorganisms in or on a suitable medium for growth and/or survival of the microbes. The microorganisms can be aerobes, anaerobes, facultative anaerobes, heterotrophs, autotrophs, photoautotrophs, photoheterotrophs, chemoautotrophs, and/or chemoheterotrophs. The microbial cell growth, maintenance or lag phase for production of any metabolites can be growing under aerobic, microaerophilic, or anaerobic conditions.

The term “fermentable sugars” as used herein refers to any one or more sugars and/or sugar derivatives that can be used as a carbon source by the microorganism, including monomers, dimers, and polymers of these compounds including two or more of these compounds. In some cases, the microorganism can break down these polymers, such as by hydrolysis, prior to incorporating the broken down material. Exemplary fermentable sugars include, but are not limited to glucose, xylose, arabinose, galactose, mannose, rhamnose, cellobiose, lactose, sucrose, maltose, and fructose. The fermentable sugars may be derived from plant materials such as corn mash.

The terms “fermentation vessel”, “vessel” “bioreactor”, “fermentation container” are used interchangeably herein and refer to the vessel in which fermentation is done for culturing of biomass/bacterial cells and production of any desired metabolite. In the current invention the desired metabolite is succinic acid.

The term “biomass” refers to the mass of the bacterial cells used for producing succinic acid. The biomass can be measured by any of the known methods in prior art such measuring OD600, dry cell weight etc.

The “growth phase” or “biomass phase” or “biomass building phase” of the bacterial cells in the fermentation bioreactor is the stage for growing the bacterial cells of the desired strain to a minimum cell weight. Growth phase is mainly an aerobic stage.

“Production phase” is the phase after the growth phase when the conditions of the fermentation vessel are altered to start the succinic acid production.

The production phase may typically after 8-26 hours of culture or when a cell density of 5-8 g-dcw/l (dcw: dry cell weight) is reached.

The term “seed culture” refers to culturing to prepare microbial cells to be subjected to the biomass phase.

The term “broth”, or “fermentation broth” as used herein, comprises all the components present in the fermentation vessel. Hence it comprises the liquid phase which contains media components and cells and optionally the product/metabolite.

The term “Clarified broth” refers to the liquid phase from which the particulate matter has been removed either by filtration or centrifugation. In general, the particulate matter is cells, also called “retentate” herein.

The term “Primary carbon source” is the carbon source without which the fermentation and succinic acid production will fail to progress. Often, sugars are the primary source of carbon for fermentation. In the current invention, sugar is the primary carbon source for energy, for microbial growth and SA production.

“Secondary source of carbon” refers to a carbon source other than glucose or sucrose or C4-C6 sugars.

[symbol] aids the fermentation system performance. The system will work without it but with reduced efficiency, especially succinic acid production will be affected by the lack of secondary carbon source. In the current invention, CO2 is the main secondary carbon source as it aids a step change in SA yield, but can not be produced entirely from CO2, in this case. In some embodiments, CO2 is used along with sodium bicarbonate as the secondary carbon source.

As used herein, the term “microaerobic conditions” refers to use of gas mixture, comprised of air and CO2, wherein CO2 is 5-35% v/v of the total gas flow and the total gas flow is lesser than 0.12 vvm, [symbol] .

As used herein, the term ‘activity reduction or enhancement’ means the activity of a gene or its encoded polypeptide is lower or higher than that of an unmodified strain or a wild-type strain.

The term “succinic acid-producing ability” used herein refers to an ability of accumulating succinic acid in a medium to such an extent that the succinic acid is collected when the bacterium is cultured in the medium.

Wildtype Corynebacterium strains like ATCC 13032, give a poor yield of succinic acid (SA). Thus, to enhance the succinic acid yield, conventionally known methods have undertaken the approach of deleting Lactate Dehydrogenase, ldh gene, while overexpressing Pyruvate Carboxylase, (pyc gene).

Examples of the coryneform bacteria include a microorganism belonging to the genus Corynebacterium, a microorganism belonging to the genus Brevibacterium, and a microorganism belonging to the genus Arthrobacter. Examples of bacterial strains that can be used for the current invention are bacteria belonging to Corynebacterium glutamicum, Brevibacterium flavum, Brevibacterium ammoniagenes, or Brevibacterium lactofermentum.

The current invention also encompasses use of any other microbes with succinic acid producing ability, using the system disclosed herein,

### Embodiments

The embodiments of the present invention encompass a process of producing succinic acid by an engineered microbial strain.

The current invention encompasses a system and a method for producing succinic acid (SA) sustainably with high yield. The invention encompasses a system and method for producing SA using CO2 as the secondary carbon source, wherein the CO2 and the biomass is recycled without having a significant negative impact on the yield of SA.

One embodiment of the current invention is a self-sustainable system for producing succinic acid, the system comprising of:
fermentation vessel (101) comprising microbial biomass,
a gas inlet (103) for sparging gas into the vessel;
a gas outlet (105) for the exhaust gas from the vessel, wherein the exhaust gas is sparged back into the vessel after passing through a gas compressor (107) to recycle the CO2;
wherein the gas sparged through the inlet is either 100% air or air mixed with 5-35% v/v of CO2 and wherein the CO2 is the secondary carbon source for succinic acid production.

In one embodiment, the pressure of gas sparged through the gas inlet (103) is 0.05 to 2 bars.

In one embodiment, CO2 is used instead of carbonate or bicarbonate salts such as sodium or potassium or ammonium or magnesium or calcium salts as the secondary carbon source for succinic acid production.

In one embodiment, the biomass in the fermenter vessel is present at 5-8 g dry cell weight/litre (dcw)/L in the fermentation vessel.

In one embodiment, liquified corn mash is used as the primary carbon source for the succinic acid production.

In one embodiment, the inlet gas is sparged at the rate of 0.03-0.11 vvm (litres/liters/min), wherein vvm is vvm=Volume of gas sparged per unit Volume of broth per Minute, Calculated by dividing the gas flow (L/m) by the Volume (L) of broth.

In one embodiment, a gas analyzer is attached to monitor the inlet and exhaust gas composition.

In one embodiment, a cell separation module (109) is connected to the fermentation vessel to separate the broth containing the succinic acid from the biomass for recycling the separated biomass into the fermentation vessel for succinic acid production.

In one embodiment, the cell separation system comprises a filtration unit or a centrifugation unit.

In one embodiment, the system comprises more than one fermentation vessels and wherein the exhaust gas from one vessel is sparged through the gas inlet of a second vessel at a pressure of 0.05 to 2 bars.

In one embodiment, the yield of succinic acid using the system disclosed herein is 0.7-0.9 g/g glucose.

One embodiment of the current invention is a method for producing succinic acid in a self-sustainable system, the method comprising the steps of:
Culturing LDH-deficient Coryneform bacterium in a fermentation vessel up to biomass of 5-8 g-dcw/L in 100% air;
Initiating succinic acid production by sparging 0.03-0.11 vvm of air mixed with 5-35% v/v CO2 gas into the vessel with the biomass grown in step (a), via a gas inlet;
recycling the exhaust gas from the gas outlet of the fermentation vessel back into the vessel via the gas inlet after passing through a gas compressor to obtain inlet pressure of 0.05 to 2 bars, to maintain SA production.

In one embodiment, the pressure of gas sparged through the gas inlet (103) is 0.05 to 2 bars. In one embodiment, CO2 is used instead of carbonate or bicarbonate salts such as sodium or potassium or ammonium or magnesium or calcium salts as the secondary carbon source for succinic acid production in the method disclosed herein.

In one embodiment, liquified corn mash is used as the primary carbon source for the succinic acid production in the method disclosed herein.

In one embodiment, a gas analyzer is attached to monitor the inlet and exhaust gas composition in the method disclosed herein.

In one embodiment, cell separation module (109) is connected to the fermentation vessel to separate the broth containing the succinic acid from the biomass for recycling the separated biomass into the fermentation vessel for succinic acid production in the method disclosed herein.

In one embodiment, the cell separation system comprises a filtration unit or a centrifugation unit in the method disclosed herein.

In one embodiment, the system comprises more than one fermentation vessels and wherein the exhaust gas from one vessel is sparged through the gas inlet of a second vessel at a pressure of 0.05 to 2 bars in the method disclosed herein.

In one embodiment, the yield of succinic acid is 0.7-0.9 g/g glucose by the method disclosed herein.

In one embodiment, the fermentation vessel contains biomass of bacterial cells, up to 5-8 g-dcw/L (dry cell weight/litre).

In one embodiment, 1 litre of the fermentation broth has 1 OD at 600 nm=0.248 gm of dry cell weight/litre.

In one embodiment, the vessel also comprises media components for supporting the bacterial growth and subsequent succinic acid production, including fermentable sugars, carbon sources, plant materials such as corn mash,

In one embodiment, the biomass is produced by culturing bacterial cells in the fermentation vessel.

In one embodiment, the system disclosed herein comprises a single fermentation vessel (101), which comprises the fermentation broth for production of succinic acid using CO2 as the secondary carbon source. The CO2 from the exhaust gas of the vessel is recycled by passing through a gas compressor, and sparged back into the vessel via the gas inlet (103) at a pressure of 0.05 to 2 bars.

In one embodiment, the biomass or cell mass in the fermentation vessel is recycled back for succinic acid production by passing through a cell separation system, collecting the cleared (clarified) broth containing succinic acid, and reusing the biomass by sending it back to the fermentation vessel.

In one embodiment, the system disclosed herein comprises more than one fermentation vessels (101), connected to each other, each vessel comprises the fermentation broth for production of succinic acid using CO2 as the secondary carbon source. The CO2 from the exhaust gas of the first vessel is recycled by passing through a gas compressor, and sparged into a second vessel via the gas inlet (103) of the second vessel, at a pressure of 0.05 to 2 bars. In one embodiment, there is a series of vessels interconnected for recycling the exhaust gas (CO2) of one vessel into a second vessel, and exhaust gas of the second vessel into a third vessel, and so on.

In one embodiment, this system can be scaled up for industrial production of succinic acid.

In one embodiment, the exhaust gas from the first fermentation vessel does not have to be passed through a gas compressor before sparging into the inlet of the second vessel.

In one embodiment, the system disclosed herein comprises more than one fermentation vessels (101), connected to each other, each vessel comprises the fermentation broth for production of succinic acid using CO2 as the secondary carbon source. The CO2 from the exhaust gas of the first vessel is recycled by passing through a gas compressor, and sparged into a second vessel via the gas inlet (103) of the second vessel, at a pressure of 0.05 to 2 bars.

In one embodiment, gas monitoring systems known in prior art are connected to the gas inlets and gas outlets in the system, to monitor the composition of the gas sparging into the vessel and the exhaust gas coming from the vessel. In one embodiment, the exhaust gas from a vessel is mixed with air to optimise the CO2 and O2 concentration before sparging it into the vessel.

In one embodiment, the gas flow, the gas concentration is regulated by mass flow controller. In one embodiment, a computer control station receiving signals from exhaust gas analyzer regulates the mass flow controller opening which are present on the lines of Air and CO2 to maintain their percentage in the compressed gas tank

The present invention addresses the problem associated with lower yield, lower productivity of succinic acid and higher cost of resources like bicarbonates by replacing them with carbon-di-oxide as a carbon source.

The present invention also includes biomass recycling for sustainability and improving the process economics.

Although the embodiments herein are described with various specific embodiments, it will be obvious for a person skilled in the art to practice the embodiments herein with modifications.

One embodiment of the invention is a process of making succinic acid by culturing an engineered microbe under microaerobic conditions. In one embodiment, the engineered bacterium is Corynebacterium glutamicum.

In one embodiment, the microaerobic conditions refer to use of a gas mixture, wherein the gas mixture is comprised of air and CO2. In one embodiment, the amount of CO2 is 5-35% v/v of the total gas flow. In one embodiment, the total gas flow is lesser than 0.12 vvm. In one embodiment, the total gas flow is in the range of 0.03 to 0.11 vvm.

In one embodiment, the transition from growth phase to production phase is done by switching the sparging of 1vvm airflow to a lower gas mixture flow in the range of 0.03-0.11 vvm. In one embodiment. the gas mixture comprises of air and CO2, wherein CO2 is in range of 5-35% v/v of the total gas flow.

In one embodiment, the transition from growth phase to production phase is done by changing the pH of the media from 7.5 to 7.8. In one embodiment the pH setpoint is maintained in the range of 7.8+/−0.5. In one embodiment the pH is maintained in the range of 7.8+/−0.3.

In one embodiment the production phase is initiated once the culture density reaches 5-8 g-dcw/l. In one embodiment the step of culturing in step a) is selected from shake flask culture, batch or fed batch culture.

In one embodiment, the engineered microbe is cultured in a bicarbonate free medium comprising a sugar source.

In one embodiment the culture in step a) is done under aerobic conditions. In one embodiment, the engineered microbe is pre-cultured.

In one embodiment the microbe is an engineered Coryneform bacterium. In one embodiment the microbe that has been genetically engineered for use in the current invention is Coryneform glutamicum.

In one embodiment the engineered microbe comprises an engineered glyoxylate pathway. In one embodiment the engineered microbe comprises a modification in the lactate dehydrogenase gene. In one embodiment the lactate dehydrogenase gene is disrupted to reduce or eliminate lactate production. In one embodiment the disruption constitutes a mutation by means of insertion, deletion or substitution. In one embodiment the disruption leads to inactivation of the gene functionality. In one embodiment the engineered microbe used herein overexpresses a pyruvate carboxylase gene. In one embodiment the overexpression of pyruvate carboxylase increases oxaloacetate pool. In one embodiment the engineered microbe comprising ldh gene disruption is Corynebacterium glutamicum. In one embodiment the engineered Corynebacterium glutamicum overexpresses pyruvate carboxylase.

In one embodiment, the engineered Coryneform bacteria, makes organic acid (OA). In one embodiment of the invention, the OA produced is in following ratio SA (succinic acid):LA (lactic acid):AA (acetic acid)::, 84:1:15 thus resulting in SA and OA yield of 0.79 and 0.94 respectively.

In one embodiment the current invention does not use NaHCO3 as a source of secondary carbon.

In one embodiment of the invention, the secondary carbon is compensated by supplying a gas mixture containing air and 5-35% v/v CO2 In one embodiment secondary carbon is compensated by supplying 20% v/v CO2. In one embodiment supplying 20% v/v CO2 culminated to SA:LA:AA:: 87:1:12 resulted in SA and OA yield of 0.87 and 1 respectively.

In one embodiment, the succinic acid yield obtained from the process of the invention is up to two fold as compared to the process without the microaerobic conditions during the production phase. In one embodiment, the succinic acid yield obtained from the process of the invention is up to 1.5 fold as compared to the process without the microaerobic conditions during the production phase.

This strategy leads to enhanced yields along with making the process more sustainable and economical as bicarbonate salts are the second most expensive raw material amongst the fermentation components. Moreover, the CO2 is recirculated, hence making this an environment friendly and sustainable process.

In one embodiment the engineered microbe has a deleted or inactivated phosphoacylase, pta gene. In one embodiment the engineered microbe has a deleted or inactivated acetate kinase, ack gene.

In one embodiment of the invention, the microbial biomass is recycled for succinic acid production. In one embodiment, the recycled biomass is directly used for SA production phase.

In one embodiment there is no loss in specific productivity of the recycled biomass

In one embodiment the recycled biomass can be used for up to 4 cycles.

In one embodiment recycling biomass for succinic acid production improves the overall economics and commercial viability of the process.

In one embodiment, the pH of the culture medium in the current invention can be adjusted by the addition of any agents such as sodium carbonate, sodium bicarbonate, potassium carbonate, potassium bicarbonate, magnesium carbonate, sodium hydroxide, calcium hydroxide, magnesium hydroxide. In one embodiment, the media pH for the biomass growth and SA production in the fermentation vessel is usually pH of 5 to 10, preferably pH of 6-8, so the pH of the culture medium can be adjusted within the above range with an alkaline material, carbonate, urea, or the like even during the reaction if required.

In one embodiment, the optimal temperature for bacterial growth to be used in the reaction of the present invention is generally in the range of 25 to 35° C. The temperature of the reaction is generally in the range of 25 to 40° C., preferably in the range of 30 to 37° C.

For culturing the bacterium, it is necessary to supply oxygen by aeration and agitation. In one embodiment, ambient air is supplied at a pressure of 0.05-2 bars for biomass growth. In one embodiment, the air is mixed with 5-35% v/v of CO2 for SA production phase, including initiation of the phase. In one embodiment, the SA is produced under microaerobic conditions in the current invention.

The foregoing description of the specific embodiments will so fully reveal the general nature of the embodiments herein that others can, by applying current knowledge, readily modify and/or adapt for various applications such as specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modifications. However, all such modifications are deemed to be within the scope of the claims.

The scope of the embodiments will be ascertained by the claims to be submitted at the time of filing a complete specification.

### EXAMPLES

### Example 1

The fermentation process of succinic acid production was categorized into two major steps:

Biomass buildup (growth phase)—in this phase the gas flow comprised 100% air.

The sequential steps involved in the biomass buildup were inoculating the microbial culture from a frozen glycerol stock, stored at (−)80° C. to 20 ml media contained in a 100 ml Erlenmeyer flask and incubated at 200 rpm, 30° C. This stage was referred to as Seed culture stage-1. Upon achieving the required optical density of 3-4 measured at 600 nm in 6-8 hrs duration, the seed-1 stage culture was used to inoculate the media volume of 200 ml contained in a 1000 ml Erlenmeyer flask and incubated at 200 rpm, 30° C. This stage 2 seed culture was used to inoculate the sterile 1000 ml media contained in fermentation vessel for the biomass buildup. (Sequential steps for biomass buildup) Seed culture-1>Seed culture-2>Fermenter—for biomass buildup.

Succinic acid production—gas flow comprising of air and CO2 or air flow only with sodium bicarbonate and sodium hydroxide solution as alkali for secondary carbon source and pH maintenance.

Biomass from step (A), is subjected to succinic acid production.

Further steps in each of these broad steps are given below:

Biomass buildup: the sequential steps followed for biomass buildup were same as mentioned previously under example 1, and the steps followed were: Seed culture-1 followed by Seed culture-2 followed by Fermenter for biomass buildup.

The strain used for evaluation of the process condition and the system performance was wild type strain ATCC13032 and its 3 mutants—(1) LDH downregulated; (2) LDH and pta-ack downregulated; (3) LDH and pta-ack downregulated with overexpression of the pyruvate carboxylase (pyc). The mutants were generated using methods as mentioned in prior art for the coryneform bacterium.

### Seed-1 Stage:

To a 100 ml Erlenmeyer flask, 20 mL of culture medium-1 comprising of 5 g/l Yeast extract, 10 g/l tryptone or peptone, 10 g/l sodium chloride either of SRL chemicals or Himedia make were used. The media solution pH adjusted to 7.2+/0.2 was sterilized at 121° C. for 20 min. Upon cooling, to this media 80 uL of pre-sterilized 50% w/v Glucose solution was added. Similarly, four number of flasks containing media were prepared. These flasks were inoculated for individual strain and its mutants respectively. The strains were wild type ATCC3032 and its (3) mutants, which are described under biomass buildup. The flask inoculated with culture were incubated in temperature-controlled incubator shaker (CIS 24 plus from Remi lab equipment, Mumbai) set at temperature 30° C. with agitation of 200 rpm. The culture was incubated for 6-18 hours, preferably for 8 hrs and then transferred to next seed stage 2 of culture propagation, referred as seed-2.

### Seed-2 Stage:

To a 1000 ml Erlenmeyer flask, 200 mL of sterile culture medium-2 comprising of 20 g/l glucose, 1.4 g/l (NH4)2SO4, 4 g/l urea 0.5 g/l K2HP04, 0.5 g/l KH2P04, 0.5 g/l MgS04·7H20, 0.5 g/l yeast extract, 0.002 g/l biotin, 0.004 g/l thiamine and 0.040 g/l FeS04·7H20 and 0.024 g/l MnS04·7H20 was inoculated with seed-1 culture, 2 to 20% v/v, to achieve an initial OD@600 nm 0.05-0.1. The media components used were either of SRL chemicals or Himedia make. The seed-2 culture was incubated at temperature-controlled incubator shaker set at temperature 30° C. and agitation of 200 rpm. The culture was incubated for 14-24 hrs, preferably for 16 hrs and then transferred to next stage of fermentation process.

### Fermenter: Biomass Buildup (Growth Phase) for Initiating Succinic Acid Production

Culture from seed stage 2, was further propagated using a fermenter, which was a 3 L jacketed glass vessel (manufacturer Eppendorf, UK and controller model no is Bioflo 120). This fermenter was equipped with controls for—temperature, gas flow, gas mixing, pH, agitation and provision for nutrient intermittent feeding, if required. The starting volume for the fermenter including the seed-2 stage culture is 1300 ml.

The fermenter medium-3 comprised of (1.2% (NH4)2SO4, 0.5% Urea, 0.08% Yeast extract, 3.5% glucose, 0.05% K2HP04, 0.05% KH2P04, 0.05% MgS04·7H20, 0.005% FeS04·7H20, 0.003% MnS04·7H20, 0.0004% Biotin, 0.0004% Thiamine). Medium (pH 7.0 to 7.8) containing 30 g (3%) of glucose, 12 g (1.2%) of (NH4)2SO4, 0.50 g (0.05%) of K2HP04, 0.50 g (0.05%) of KH2P04, 0.5 g (0.05%) of MgS04·7H20, 0.5 g (0.05%) of yeast extract, 0.002 g (0.0002%) biotin, 0.002 g (0.0004%) thiamine and 0.040 g (0.004%) FeS04·7H20 and 0.024 g (0.0024%) MnS04·7H20 in 1000 ml of a total volume was especially preferred.

The glass fermenter was incubated at 30° C.+/−0.5 and the pH setpoint of 7.5+/−0.3 was maintained using alkali and acid solution of 5N sodium hydroxide and 2.5 N sulphuric acid respectively. Gas flow, comprising only of air, was sparged 0.5-1.2 vvm, preferably 1 vvm and agitation was ramped up from 300-700 rpm for the duration of 0-10 hrs, controlled via time elapsed profile versus agitation. During this period the microbial biomass reaches 5-8 g-dcw/L (dry cell weight/litre).

### Succinic Acid Production

Upon achieving desired biomass of 5-8 g-dcw/L in the above-mentioned step of biomass buildup, the succinic acid production was initiated by making below changes:

Switch gas flow from 1 vvm of air to the gas flow of 0.03 vvm comprising of 100% v/v of air. The gas flow was not changed, increased or decreased, during the batch progress (fermentation batch/reaction progress). It was passed through the sparger in both the phases-biomass buildup and succinic acid production.

Switch pH setpoint from 7.5+/−0.3 to 7.8+/−0.3, controlled by addition of alkali reagent, a mixture of sodium bicarbonate and sodium hydroxide prepared by adding to water the 221 g/L and 80 g/L of each of the reagent respectively and making up final volume to 1000 ml.

Agitation speed fixed at 400 rpm.

Glucose solution total volume of 230 ml, was added as bolus at different time points. Glucose solution used was prepared as stock solution of 600 g/kg (60% w/v), autoclaved in advance at 121° C., 20 min and cooled to room temperature.

Analysis of the fermented broth sample for glucose, succinic acid, lactic acid, acetic acid was carried out after centrifuging the sample at 13000 rpm for 5 min and the supernatant was diluted and filtered through 0.2 u before injecting into the HPLC column. The respective compound concentrations were calculated taking into consideration the standard curve and the appropriate dilution factor. The compounds for building the reference standard calibration curve were procured from the chemical company Sigma Aldrich.

The total fermentation process time of 70-80 hrs comprise of 8-24 hrs of biomass buildup and succinic acid production phase of 40-60 hrs in the fermenter vessel.

As part of this work, the strains with different gene either downregulated or overexpressed were evaluated under above mentioned process parameters and then tested under condition where sodium bi-carbonate was replaced by carbon di-oxide (CO2) purging into fermentation broth along with the air.

To compare the impact of bicarbonate salts and CO2 as secondary carbon source on the SA production, the wild strain and its 3 mutants—(mutant 1) LDH downregulated, (mutant 2) LDH and pta-ack downregulated, (mutant 3) LDH and pta-ack downregulated with overexpression of the pyruvate carboxylase (mutant were generated using methods reported in prior art for the coryneform bacterium; Ref 3: Schafer A et al)—were subjected to SA production using sodium bicarbonate. These were control trials for SA production by using bicarbonate. Results of these trials were compared against the trials where sodium bicarbonate was replaced with CO2. The results of the control trial where sodium bicarbonate was used are summarized below in Table 1.

### Table-1: Yield of Succinic Acid Obtained for Coryneform glutamicum Wild Type and its Mutants Under Sodium Bicarbonate as the Secondary Carbon Source

| | | | Yield g/g Glucose | | |
|---|---|---|---|---|---|
| Strain | Gas flow, vvm | Air % | SA | AA | LA |
| Wild | 0.031 | 100 | 0.21 | 0.04 | 0.81 |
| Mutant (1) | 0.031 | 100 | 0.72 | 0.16 | 0.01 |
| Mutant (2) | 0.031 | 100 | 0.76 | 0.17 | 0.01 |
| Mutant (3) | 0.031 | 100 | 0.79 | 0.14 | 0 |

### Example 2

Based on the results obtained in example 1, the mutant (3) which showed highest SA yield amongst the tested mutants was selected for evaluation under CO2 as secondary carbon source. In this trial no bicarbonate salts were used.

Succinic acid production was done under 0.031 vvm of gas comprising of air and CO2 (20% v/v of gas flow). The alkali reagent used was sodium hydroxide solution, 4.5 N, prepared by adding 180 gram of sodium hydroxide pellets to 800 ml of water and the final volume was made up to 1000 ml. In this trial bicarbonate salts were not used.

The biomass buildup was done by the same method as given in example 1.

The succinic acid production was initiated by:

Switching the gas flow from 1 vvm of air to the gas flow of 0.031 vvm comprising of air and carbon di-oxide (20% v/v of the total gas flow). The gas flow is not changed, increased or decreased, during the batch progress. It is passed through the sparger in both the phases-biomass buildup and succinic acid production.

Switching pH setpoint from 7.5+/−0.3 to 7.8+/−0.3, and it is controlled by addition of alkali reagent, a sodium hydroxide solution prepared by adding to water, 80 g/L, 160 g/L, 200 g/L, 250 g/L and 280 g/L, preferably 180 g/L of the reagent and making up final volume to 1000 ml.

### TABLE-2: Yield of Succinic Acid Obtained With Mutant (3) of Coryneform glutamicum With CO2 as the Secondary Carbon Source

| | | | Yield g/g Glucose | | |
|---|---|---|---|---|---|
| Gas flow, vvm | Air % v/v | CO2 % v/v | SA | AA | LA |
| 0.031 | 80 | 20 | 0.87 | 0.13 | 0.00 |

Comparison of the results from example 1 and 2, indicate improved performance of the mutant (3) when CO2 was used as secondary carbon source instead of bicarbonate salt.

### Example 3

### Comparison of Mutant (3) Performance for Succinic Acid Production Under Different Conditions as Mentioned Below

The biomass buildup was done in similar manner as mentioned in example 1, followed by succinic acid production under below conditions
Condition 1: under sodium bicarbonate and sodium hydroxide solution as alkali reagent with air flow
Condition 2: under gas flow comprising of CO2 (20% v/v) and air (80% v/v) and alkali reagent was sodium hydroxide solution. No bicarbonate salt was used.
Condition 3: under combination of condition 1 and 2, as described above.

### Table-3: Yield Comparison of Succinic Acid for Mutant (3) of Coryneform glutamicum Under Different Condition of Secondary Carbon Source. The Conditions as Described Above From Serial No i-iii

| Organic | | | |
|---|---|---|---|
| Acid | Condition 1 | Condition 2 | Condition 3 |
| SA | 0.78 | 0.87 | 0.84 |
| LA | 0.00 | 0.00 | 0.01 |
| AA | 0.13 | 0.15 | 0.22 |

### Example-4

### Biomass Recycle for Succinic Acid Production

Microbial biomass was re-used for succinic acid production. Biomass buildup was done for cycle-1 and succinic acid production was done in similar manner as in example 2. Upon completion of succinic acid production for the cycle-1, the biomass was separated from the fermented broth by centrifugation or tangential flow filtration unit. The separated biomass was resuspended in the medium 4 and subjected directly to succinic acid production as per example 1. This process of recycle was repeated till drop of 80% or more was observed in productivity of cells for the succinic acid production. The result is shown in Table-4

The medium 4 comprises of (0.5% (NH4)2SO4, 3.5% glucose, 0.05% K2HP04, 0.05% KH2P04, 0.05% MgS04·7H20, 0.005% FeS04·7H20, 0.003% MnS04·7H20, 0.0004% Biotin, 0.0004% Thiamine). in 1000 ml of a total volume was especially preferred.

### Table-4: Yield of Succinic Acid Obtained for Mutant (1) of Coryneform glutamicum With CO2 as Only Secondary Carbon Source, Using Recycled Biomass up to 4 Cycles

| Strain | Cycle | % change in Succinic acid production efficiency |
|---|---|---|
| Mutant (1) | Cycle_1 | 100 |
| | Cycle_2 | 96.1 |
| | Cycle_3 | 88.0 |
| | Cycle_4 | 86.7 |

Biomass recycle eliminated the requirement of running the biomass buildup cycle for each succinic acid production cycle.

### Example 5

### Liquified Corn Mash was Used for the Succinic Acid Production and the Biomass was Recycled

Coarse grinded corn powder was subjected to industrially relevant steps of liquefaction and saccharification to obtain a syrup having glucose. The sample was centrifuged, and the supernatant was analysed using HPLC for glucose mass buildup was done in similar manner as in example 1.

Succinic acid production was initiated by resuspending the biomass equivalent of 5-8 g-dcw/L in the media that comprised of 575 ml of corn mash (containing glucose 267 g/L), (0.4% (NH4)2SO4, 0.05% K2HP04, 0.05% KH2P04 with remaining conditions same as in example 1.

### TABLE-5: Yield of Succinic Acid Using Corn Mash as Additional Primary Carbon Source

| | | Yield g/g Glucose | | |
|---|---|---|---|---|
| Strain | Media- Succinic acid production | SA | AA | LA |
| Mutant (1) | same in Example 1 | 0.72 | 0.16 | 0.03 |
| Mutant (1) | Corn mash | 0.72 | 0.18 | 0.02 |

The biomass from the succinic acid production using corn mash was further subjected to biomass recycling for succinic acid production, in similar manner as in example 4, except that corn mash was used instead of glucose solution. Results similar to example 3 were obtained.

### Example 6

### Gas Recycling was Enabled During Succinic Acid Production in Fermenter Train by Connecting the Gas Flow Outlet From Fermenter 1 to the Gas Inlet of Fermenter 2

The biomass buildup was same as given in example 1.

The impact of gas recycling for SA production was compared under the below mentioned conditions

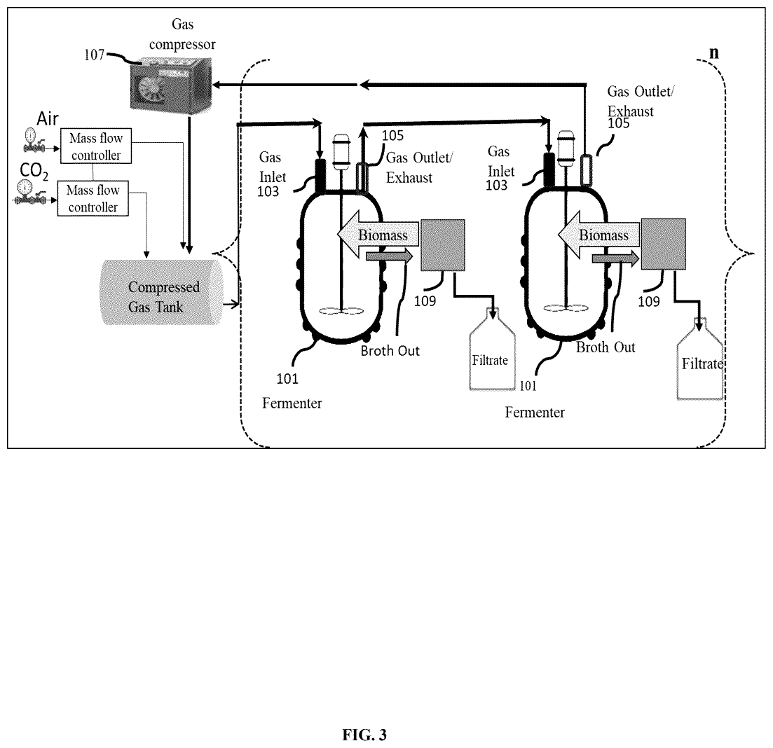

Succinic acid production with bicarbonate salt: The succinic acid production was done as mentioned in example 1 using sodium bicarbonate however the fermenter setup was as shown in FIG. 3, which was different from the setup in experiment 1. Here the outlet gas from fermenter-1, referred as F1, was used as input gas for the Fermenter-2, referred as F2, during the succinic acid production phase, enabling the recycling of the gas. Result summarized in below table.

Succinic acid production with CO2: The succinic acid production was done as mentioned in example 2, under gas flow comprising of air and CO2 (5-30% v/v) and the alkali reagent used was sodium hydroxide. No bicarbonate was used. The fermenter setup is same as in (6 a), enabling the recycling of the CO2 gas.

### TABLE-6: Yield Comparison of Succinic Acid During Gas Recycle in Fermenter Train

| | | Under Gas flow comprising of | |
|---|---|---|---|
| Under Bicarbonate salt | | CO2 (20% v/v) and air | |
| Fermenter | Yield SA_g/g_Glu | Fermenter | Yield SA_g/g_Glu |
| F1 | 0.78 | F1 | 0.87 |
| F2 | 0.79 | F2 | 0.87 |

1. A self-sustainable system for producing succinic acid, the system comprising of a
a. fermentation vessel (101) comprising microbial biomass,
b. a gas inlet (103) for sparging gas into the vessel;
c. a gas outlet (105) for the exhaust gas from the vessel, wherein the exhaust gas is sparged back into the vessel after passing through a gas compressor (107) to recycle the CO2;
wherein the gas sparged through the inlet is either 100% air or air mixed with 5-35% v/v of CO2 and wherein the CO2 is the secondary carbon source for succinic acid production.

2. The system of claim 1, wherein the pressure of gas sparged through the gas inlet (103) is 0.05 to 2 bars.

3. The system of claim 1, wherein CO2 is used instead of bicarbonate salts of sodium, potassium, ammonium, magnesium or calcium as the secondary carbon source for succinic acid production.

4. The system of claim 1, wherein the biomass is present at 5-8 g dry cell weight/litre (dcw)/L in the fermentation vessel.

5. The system of claim 1, wherein liquified corn mash is used as the primary carbon source for the succinic acid production.

6. The system of claim 1, wherein the inlet gas is sparged at the rate of 0.03-0.11 vvm (litres/liters/min).

7. The system of claim 1, wherein a gas analyzer is attached to monitor the inlet and exhaust gas composition.

8. The system of claim 1, wherein a cell separation module (109) is connected to the fermentation vessel to separate the broth containing the succinic acid from the biomass for recycling the separated biomass into the fermentation vessel for succinic acid production.

9. The system of claim 8, wherein the cell separation system comprises a filtration unit or a centrifugation unit.

10. The system of claim 1, wherein the system comprises more than one fermentation vessels and wherein the exhaust gas from one vessel is sparged through the gas inlet of a second vessel at a pressure of 0.05 to 2 bars.

11. The system of claim 1, wherein the yield of succinic acid is 0.7-0.9 g/g glucose.

12. A method for producing succinic acid in a self-sustainable system, the method comprising the steps of:
Culturing LDH-deficient Coryneform bacterium in a fermentation vessel up to biomass of 5-8 g-dcw/L in 100% air;
b. Initiating succinic acid production by sparging 0.03-0.11 vvm of air mixed with 5-35% v/v CO2 gas into the vessel with the biomass grown in step (a), via a gas inlet;
c. recycling the exhaust gas from the gas outlet of the fermentation vessel back into into the vessel via the gas inlet after passing through a gas compressor to obtain inlet pressure of 0.05-2 bars, to maintain SA production.

13. The method of claim 12, wherein the pressure of gas sparged through the gas inlet (103) is 0.05 to 2 bars.

14. The method of claim 12, wherein CO2 is used instead of bicarbonate salts such as sodium or potassium or ammonium or magnesium or calcium salts as the secondary carbon source for succinic acid production.

15. The method of claim 12, wherein liquified corn mash is used as the primary carbon source for the succinic acid production.

16. The method of claim 12, wherein a gas analyzer is attached to monitor the inlet and exhaust gas composition.

17. The method of claim 12, wherein a cell separation module (109) is connected to the fermentation vessel to separate the broth containing the succinic acid from the biomass for recycling the separated biomass into the fermentation vessel for succinic acid production.

18. The method of claim 12, wherein the cell separation system comprises a filtration unit or a centrifugation unit.

19. The method of claim 12, wherein the system comprises more than one fermentation vessels and wherein the exhaust gas from one vessel is sparged through the gas inlet of a second vessel at a pressure of 0.05 to 2 bars.

20. The method of claim 12, wherein the yield of succinic acid is 0.7-0.9 g/g glucose.
